(19) United States
(12) Patent Application Publication
(10) Pub. No.: US 2021/0144971 A1
(43) Pub. Date: May 20, 2021

(54) WEARABLE INSTRUMENT, MEASURING INSTRUMENT, WEARABLE INSTRUMENT SET, AND ADJUSTMENT METHOD

(71) Applicants: Sharp Kabushiki Kaisha, Sakai City, Osaka (JP); UNIVERSITY PUBLIC CORPORATION OSAKA, Osaka-shi, Osaka (JP)

(72) Inventors: Tetsuya HAYASHI, Sakai City (JP); Azusa NAKANO, Sakai City (JP); Hiroshi SAKAYA, Sakai City (JP); Shunsuke SHIMAMURA, Sakai-shi (JP); Terumasa SHIMADA, Sakai-shi (JP)

(21) Appl. No.: 16/636,665

(22) PCT Filed: Aug. 7, 2018
(86) PCT No.: PCT/JP2018/029682

§ 371 Date: Feb. 5, 2020

(30) Foreign Application Priority Data
Aug. 10, 2017 (JP) 2017-156090

(51) Int. Cl. A01K 29/00; A01K 27/00

(52) U.S. Cl. CPC A01K 29/005; A61B 5/6822; A01K 27/001

(57) ABSTRACT

Provided are a wearable instrument and other things capable of setting suitable tightness. A wearable device includes the following: a collar; a belt having one end connected to the collar, and extending to the animal's back via a dog's armpit when the wearable device is attached to the dog; and a joint where the belt length is adjusted. The belt has a wearable-device index indicated for the length adjustment with the joint, the wearable-device index corresponding to a measuring-instrument index indicated on an index belt included in a measuring instrument.

### TECHNICAL FIELD

The present disclosure relates to a wearable instrument for a living body, particularly for an animal covered with fur. The present disclosure also relates to a measuring instrument that measures a suitable length of the belt of the wearable instrument, a wearable instrument set including the wearable instrument and measuring instrument, and a method for adjusting the wearable instrument using the measuring instrument.

### BACKGROUND ART

Daily healthcare is widely recognized as being important for precautions and countermeasures against lifestyle diseases. As is the case with humans (i.e., pet owners herein), daily healthcare has been increasingly recognized as being important for companion animals (i.e., pets), thus increasing a demand for a pet owner to measure the biological information of his/her pet readily.

Patent Document 1 discloses an example of measuring the biological information of a pet. Patent Document 1 describes a body-fat measuring instrument for pets. This body-fat measuring instrument includes a securing component for keeping each of electrodes pressed against armpits and other sites of a pet, and also includes a tightness adjustment component for adjusting the tightness against the animal wearing the body-fat measuring instrument.

### CITATION LIST

### Patent Literature

Patent Document 1: Japanese Patent Application Laid-Open No. 2005-27661, published on Feb. 3, 2005

### SUMMARY OF INVENTION

### Technical Problem

In the body-fat measuring instrument for pets disclosed in Patent Document 1, the tightness against an animal can vary depending on a user who attaches the body-fat measuring instrument to the animal. Such tightness variations unfortunately lead to a problem, such as (i) excessively great tightness can inflict suffering on the animal, or (ii) excessively small tightness can fail to bring the electrodes into proper contact with the animal.

It is an object of one aspect of the present disclosure to provide a wearable instrument and other things that facilitate setting of suitable tightness.

### Solution to Problem

To solve the above problem, one aspect of the present disclosure provides a wearable instrument for an animal. The wearable instrument includes the following: a collar attachable to the neck of the animal; and a belt portion having one end joined to the collar, the belt portion extending to the back of the animal via an armpit of the animal when the wearable instrument is attached to the animal The belt portion includes an adjustment portion where the length of the belt is adjusted. The belt portion is at least partly stretchable. The belt portion includes a first index indicated for the length adjustment with the adjustment portion. The first index corresponds to a second index indicated on an index belt included in a measuring instrument corresponding to the wearable instrument.

Another aspect of the present disclosure provides a measuring instrument. The measuring instrument includes the following: a measuring collar attachable to the neck of an animal; an index belt having a first end joined to the measuring collar, the index belt including a second index indicated in accordance with the distance from the joint between the measuring collar and the first end; and a collar accessory joined to the measuring collar, the collar accessory being disposed at the back of the animal when the measuring instrument is attached to the animal.

### Advantageous Effect of Invention

The wearable instrument, measuring instrument, and other things according to the aspects of the present disclosure enable setting of suitable tightness without reliance on a user.

### BRIEF DESCRIPTION OF DRAWINGS

FIG. 1(a) is a perspective view of the configuration of a wearable instrument according to a first embodiment. FIG. 1(b) is a plan view of the configuration of a measuring instrument according to a second embodiment.

FIG. 2 shows the measuring instrument shown in FIG. 1(b) in use.

FIG. 3(a) shows a region of a belt portion between an electrode and a joint. FIG. 3(b) shows an index belt.

FIG. 4(a) is a table showing the normal length of a stretchable belt for each size of the wearable instrument, and also showing, for each size of the instrument, length necessary for elongating the stretchable belt by 13.3%. FIG. 4(b) is a table showing an example of the relationship in length between small sections for each size of the wearable instrument and small sections of the measuring instrument of corresponding size.

FIG. 5 shows how the wearable instrument according to the first embodiment is attached to a dog. FIG. 5(a) is a diagram in the middle of the attachment, and FIG. 5(b) is a diagram after the completion of the attachment.

FIG. 6 is a perspective view of the configuration of a measuring instrument according to a second embodiment.

FIG. 7 is a perspective view of the configuration of a wearable instrument according to a third embodiment.

### DESCRIPTION OF EMBODIMENTS

The following describes an instance where a target animal is a dog. The target animal is not limited to a dog. The target animal may be (i) a companion animal (e.g., a cat, rabbit, ferret, monkey or hamster), (ii) a farm animal (e.g., a horse, cow, pig, sheep, or goat), or (iii) a tiger, lion, or other animals.

### First Embodiment

One embodiment of the present disclosure will be detailed. The present embodiment describes a wearable instrument 1 for measuring the cardiac electrical activity of the dog and other things, and describes a measuring instrument 2 that is used to adjust the length of the belt portion of the wearable instrument 1. The wearable instrument 1 and the measuring instrument 2 are included in a wearable instrument set according to the present embodiment.

### Wearable Instrument 1

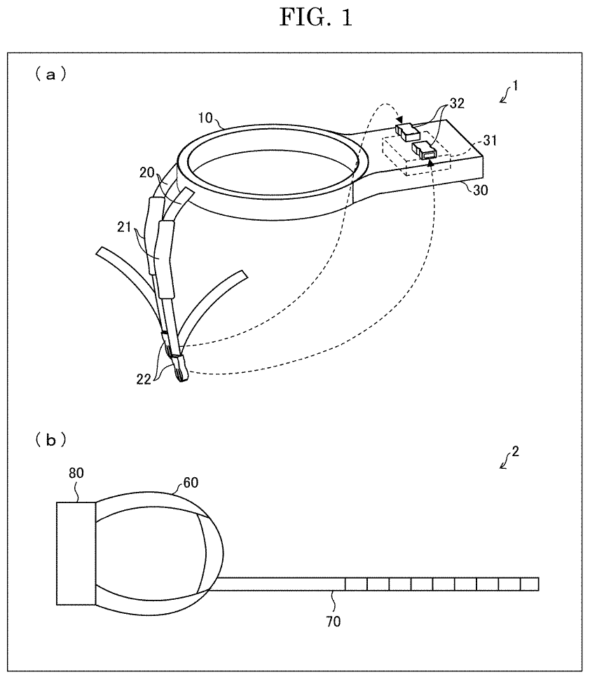

FIG. 1(a) is a perspective view of the configuration of the wearable instrument 1 according to the present embodiment. The wearable instrument 1 is used to measure, for instance, the cardiac electrical activity of a dog. Biological information to be measured by the wearable instrument 1 is not limited to cardiac electrical activity. The biological information may be, for instance, pulse wave, perspiration rate, heart rate, or body fat percentage. As shown in FIG. 1, the wearable instrument 1 includes a collar 10, belt portions 20, and a casing 30.

The collar 10 is an annular member attachable to the dog's neck. The collar 10 needs to be annular during attachment to the dog, and may have a seemingly annular shape formed by, for instance, a plurality of bands joined together. The collar 10 may be made of any material, such as natural leathers, artificial leathers, resins, natural fibers, or chemical fibers. The collar 10 may further include an adjustment portion for adjusting the length of the collar 10 in accordance with the neck diameter of the dog.

Each belt portion 20 has one end joined to the collar 10. When the dog is wearing the wearable instrument 1, the belt portions 20 extend to the dog's back via dog's armpits. The wearable instrument 1 includes two belt portions 20 corresponding to the dog's respective right and left arms. Each belt portion 20 includes an electrode 21 and a joint 22. In some embodiment, the wearable instrument 1 includes any number of belt portions 20 in accordance with what is measured.

The electrodes 21 are used to measure the cardiac electrical activity of dog as biological information. The electrodes 21 are made of conductive cloth for instance. The electrodes 21 are nearly cylindrical so as to be easily fitted in the dog's arms.

The electrodes 21 are disposed at the axillae of the dog with the wearable instrument 1 attached thereto. Dog's axillae are relatively less covered with fur in the body. Accordingly, bringing the electrodes 21 into contact with the axillae enables the cardiac electrical activity to be measured without fur shaving or application of a conductive gel. The axillae are less likely to come into contact with a floor, wall, or other things. Hence, a floor or wall contact is less likely to change the position of the electrodes 21 and is thus less likely to hinder correct measurement.

In the present embodiment, the two electrodes 21 are included in the respective belt portions 20. It is noted that any number of electrodes 21 may be provided according to what is measured by the wearable instrument 1, and that different belt portions 20 may include different number of electrodes 21.

Each joint 22 is a member for detachably joining the other end of the corresponding belt portion 20 to the casing 30. In the wearable instrument 1, each belt portion 20 forms an annular portion between part of the collar 10 and part of the casing 30. The joint 22 in the present embodiment is a buckle. The length of the belt portion 20 is adjustable by regulating a location in which the body of the belt portion 20 is folded back at the joint 22. In this way, the joints 22 serve as adjustment portions for adjusting the lengths of the belt portions 20.

It is noted that such an adjustment portion, where the length of the belt portion 20 is adjusted, may be provided separately from the joint 22. In this case, examples of the adjustment portion include an adjuster, a pin buckle, a button, a hook-and-loop fastener, a needle (e.g., safety pin), and a reel for winding a belt in. Each of these example adjustment portions is a typical size (i.e., length herein) adjustment mechanism that is used in clothes and other things for size (i.e., length herein) adjustment. A user adjusts the lengths of the belt portions 20 using such a mechanism.

The casing 30 contains a measuring device 31 for measuring the cardiac electrical activity of the dog and other things. The measuring device 31 is connected to the electrodes 21 via conductive wires not shown. The casing 30 includes, on its exterior, joints 32 connectable to the respective joints 22 of the belt portions 20. It is noted that the joints 32 may be disposed in the collar 10 instead of the casing 30.

The measuring device 31 includes a sensor substrate, an infrared sensor, and a buttery, which are not shown. Mounted on the sensor substrate are a signal processing circuit for cardiac electrical activity, an acceleration sensor, a temperature-humidity sensor, a microcomputer, and a Bluetooth (registered trademark) low energy (BLE) module. The BLE module includes an antenna for data transmission to the outside. It is noted that a communication module other than the BLE module may be mounted on the sensor substrate. The infrared sensor is used to measure the temperature of the dog's body surface. The buttery supplies power to the sensor substrate and infrared sensor.

The signal processing circuit for cardiac electrical activity acquires a potential from the electrodes 21 as a signal indicating the cardiac electrical activity of the dog. The signal processing circuit for cardiac electrical activity also amplifies the acquired signal, and filters the signal for noise removal.

The piece of data containing the dog's cardiac electrical activity processed by the signal processing circuit for cardiac electrical activity and the pieces of data acquired from the other sensors (i.e., dog's body surface temperature, acceleration, and temperature and humidity of the environment) are transmitted to and stored in another terminal, such as a smartphone or a tablet, by the BLE module. It is noted that these data pieces may be stored in a Cloud server via a smartphone or other terminals. Alternatively, the sensor substrate may include a WiFi (registered trademark) module. In this case, these data pieces may be stored in the Cloud server directly from the sensor substrate, not via another terminal.

It is also noted that the measuring device 31 does not have to include the signal processing circuit for cardiac electrical activity, and may transmit the potential acquired from the electrodes 21, as is, to a different terminal. In this case, signal amplification and signal filtering are executed in this different terminal.

### Measuring Instrument 2

FIG. 1(b) is a plan view of the configuration of the measuring instrument 2 according to the present embodiment. The measuring instrument 2 is used to adjust the lengths of the belt portions of the wearable instrument 1. As shown in FIG. 1(b), the measuring instrument 2 includes a measuring collar 60, an index belt 70, and a collar accessory 80.

The measuring collar 60 is an annular member attachable to the dog's neck. The measuring collar 60, as is the case with the collar 10, needs to be annular during attachment to the dog. The measuring collar 60 is made of a material similar to that of the collar 10. The measuring collar 60, as is the case with the collar 10, may further have an adjustment portion for adjusting the length of the measuring collar 60 according to the diameter of the dog's neck.

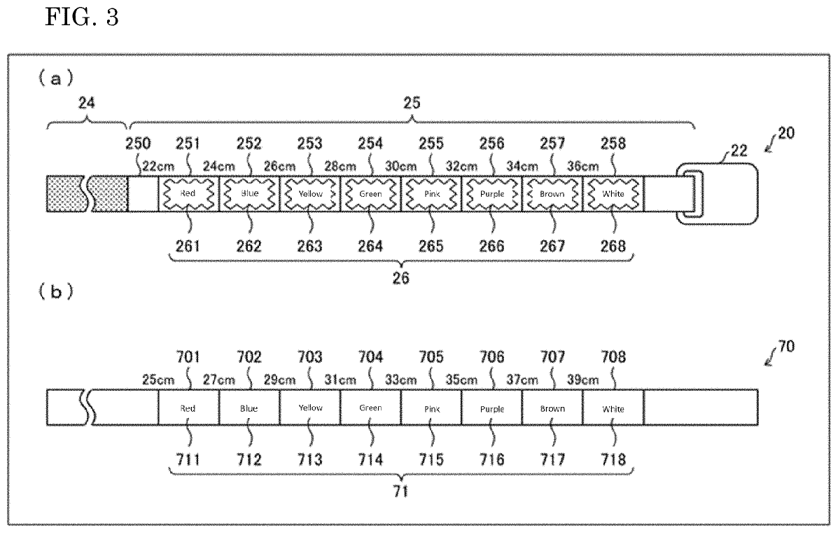

The index belt 70 has a first end joined to the measuring collar 60. The index belt 70 in its entirety is made of a relatively less stretchy material (e.g., cloth or nylon). Reference is made to FIG. 3(b). The index belt 70 includes a measuring-instrument index 71 (i.e., second index) indicated in accordance with the distance from the joint between the index belt 70 and the measuring collar 60.

The collar accessory 80 is a member indicating a location for checking a suitable graduation of the measuring-instrument index 71. A graduation herein is not only a dot and line, but also a character, a numeral, a graphic, a sign, a color, or a combination thereof, and means a mark serving as a guide for length adjustment. The collar accessory 80 is joined to the measuring collar 60. The collar accessory 80 is disposed at the dog's back when the measuring instrument 2 is attached to the dog.

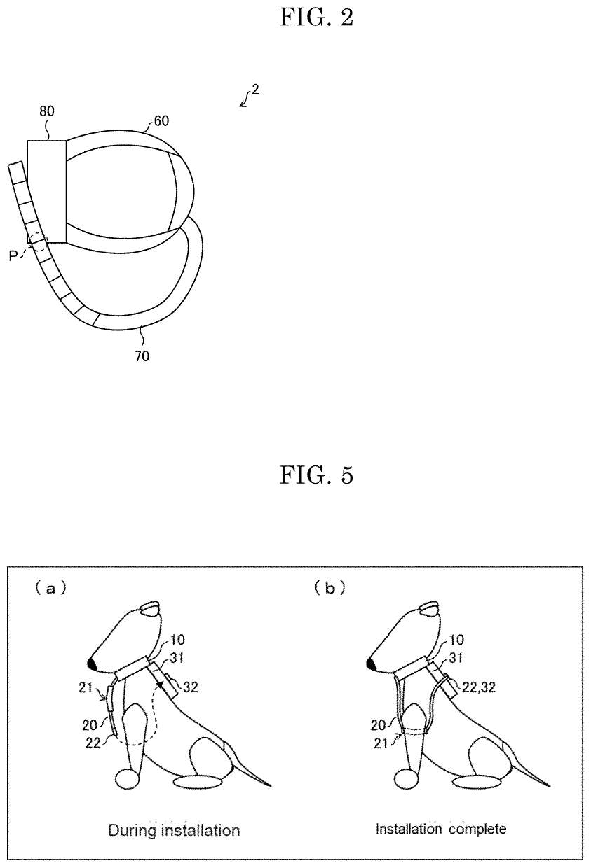

FIG. 2 shows the measuring instrument 2 in use. When using the measuring instrument 2, the user attaches the measuring collar 60 to the dog's neck, followed by pulling the index belt 70 up via the dog's armpits to bring the index belt 70 into contact with the collar accessory 80 located at the dog's back, as shown in FIG. 2. At this time, the user checks the graduation in a location P intersecting with the outer edge on the side of the collar accessory 80. This outer edge on the side means an outer edge that, when viewed from the back of the dog wearing the measuring instrument 2, extends along the dog's backbone. The index belt 70, which is made of a less stretchy material, is less likely to generate an index deviation resulting from unnecessary expansion and contraction in index checking.

### Belt Portion 20 and Index Belt 70

FIG. 3(a) shows a region of the belt portion 20 between the electrode 21 and the joint 22. As shown in FIG. 3(a), the region of the belt portion 20 includes a stretchable belt 24 having stretchability, and a non-stretchable belt 25 having no stretchability when compared to the stretchable belt 24. In other words, the belt portion 20 is at least partly stretchable. The elastic force of the stretchable belt 24 brings the electrode 21 into contact with the dog's skin.

The stretchable belt 24 is made of a material with large stretchability (i.e., elastic material), such as rubber. The non-stretchable belt 25 is made of a material with smaller stretchability than that of the stretchable belt 24 (e.g., cloth and nylon). In the belt portion 20, the stretchable belt 24 is closer to the collar 10 than the non-stretchable belt 25. The electrode 21 is disposed on the stretchable belt 24. The stretchable belt 24 has a length such that the electrode 21 is in contact with the axilla of the dog when the wearable instrument 1 is attached to the dog.

The belt portion 20 has a wearable-instrument index 26 (i.e., first index) indicated for the length adjustment with the joint 22. The wearable-instrument index 26 has marks that are displayed to correspond to the measuring-instrument index 71 indicated on the index belt 70.

The wearable-instrument index 26 in the present embodiment includes, as graduations, marks 261 to 268 displayed in the length direction of the belt portion 20. The non-stretchable belt 25 has eight small sections 251 to 258 that are set in the length direction. The marks 261 to 268 are indicated so as to respectively correspond to the small sections 251 to 258. The small sections 251 to 258 and the marks 261 to 268 are arranged in this order starting from a location of the non-stretchable belt 25 close to the collar 10.

The marks constituting the wearable-instrument index 26 and their display order are the same as a plurality of marks constituting the measuring-instrument index 71 and their display order. Nevertheless, the marks constituting the wearable-instrument index 26 and the marks constituting the measuring-instrument index 71 do not have to have the same shape and same color. Both sets of marks need to be similar enough for the user to be able to recognize that the marks constituting the wearable-instrument index 26 correspond one-to-one to the marks constituting the measuring-instrument index 71.

The shape of the marks 261 to 268 is non-limiting, and may have any shape. In the present embodiment, the marks 261 to 268 are different from each other in color. The marks 261 to 268 may have their colors indicated in FIG. 3(a) and as follows for instance:

The mark 261 is red,
The mark 262 is blue,
The mark 263 is yellow,
The mark 264 is green,
The mark 265 is pink,
The mark 266 is purple,
The mark 267 is brown, and
The mark 268 is white.
It is preferable that the combination of the adjacent colors of the marks 261 to 268 be readily distinguishable visually. For instance, the combination may include a combination of cool and warm colors.

It is noted that the wearable-instrument index 26 may include any set of marks other than the marks 261 to 268 with different colors from each other. To be specific, the wearable-instrument index 26 may include a plurality of marks with different shapes, not colors, or may include a plurality of marks with different colors and different shapes. The aforementioned marks may be indicated by, for instance, distinguishing the material or knitting of the belt portion 20, may be indicated by coloring or needlework, or may be indicated by forming asperities. The number of small sections and the size of each small section are non-limiting, and the small sections do not have to have the same size.

The non-stretchable belt 25 of the belt portion 20 in FIG. 3(a) has an adjustment region 250 located closer to the collar 10 than the small section 251. The adjustment region 250 is set in the production of the belt portion 20 and is set for adjusting the length of the belt portion. For elongating the belt portion 20, the adjustment region 250 is set to be long. The adjustment region 250, however, does not have to be disposed on the belt portion 20.

The region labeled as the non-stretchable belt 25 of the belt portion 20 in FIG. 3(a) may be made of a material with large stretchability, as is the case with the stretchable belt 24.

FIG. 3(b) shows the index belt 70 of the measuring instrument 2. As earlier described, the measuring-instrument index 71 is displayed on the index belt 70. As shown in FIG. 3(b), the index belt 70 has eight small sections 701 to 708 in its length direction. The measuring-instrument index 71 in the present embodiment includes colors 711 to 718 respectively disposed in the small sections 701 to 708 and respectively having the same colors as the marks 261 to 268. It is noted that the measuring-instrument index 71, although including the colors 711 to 718 in the present embodiment, may include one of the aforementioned various graduations. The aforementioned graduations may be indicated by distinguishing material or knitting, may be indicated by coloring or needlework, or may be indicated by forming asperities.

In FIG. 3(a), each of the lengths recited at the respective boundaries between the small sections 251 to 258 indicates the distance between the end of the belt portion 20 joined to the collar 10 and the corresponding boundary. In FIG. 3(b), each of the lengths recited at the respective boundaries between the small sections 701 to 708 indicates the distance between the end of the index belt 70 joined to the measuring collar 60 and the corresponding boundary. As shown in FIG. 3(a) and FIG. 3(b), distance (i), i.e., the distance between the end of the belt portion 20 joined to the collar 10 and each of the small sections 251 to 258, is different from distance (ii), i.e., the distance between the end of the index belt 70 joined to the measuring collar 60 and each of the small sections 701 to 708.

To be specific, as shown in FIG. 3(a), the small sections 251 to 258 with the stretchable belt 24 not elongated are eight small sections formed by equally dividing a region having a length of 22 cm or more and less than 38 cm starting from the end of the belt portion 20 joined to the collar 10. As show in FIG. 3(b), the small sections 701 to 708 are eight small sections formed by equally dividing a region having a length of 25 cm or more and less than 41 cm starting from the end of the index belt 70 joined to the measuring collar 60. In other words, the small sections corresponding to the respective marks 261 to 268 are each 3 cm shorter in length starting from the end of the belt portion 20 than the small sections corresponding to the respective colors 711 to 718.

As earlier described, the belt portion 20 includes the stretchable belt 24. Elongating the stretchable belt 24 enables distance (i), i.e., the distance between the end of the belt portion 20 joined to the collar 10 and the small section with a suitable mark, to be equal to distance (ii), i.e., the distance between the end of the index belt 70 joined to the measuring collar 60 and the small section with a suitable mark.

When the user sets the joint 22 at the mark corresponding to the graduation checked with the measuring instrument 2, the length of the belt portion 20 is adjusted to be suitable for the dog's size. When the user puts, into the dog, the wearable instrument 1 that has undergone such length adjustment, the stretchable belt 24 is 3 cm elongated for instance. In any of the colors 711 to 718 being a graduation checked with the measuring instrument 2, attaching, to the dog, the wearable instrument 1 with the joint 22 set at the mark corresponding to the graduation elongates the stretchable belt 24 by a fixed length, e.g., 3 cm. Thus, in any of the colors 711 to 718 being a graduation checked with the measuring instrument 2, the stretchable belt 24 produces, likewise, a fixed degree of elastic force.

Elongating the stretchable belt 24 by a suitable ratio of length with respect to its normal length enables the electrode 21 to come into contact with the dog's body with suitable pressure in the wearable instrument 1. In the present embodiment, elongating the stretchable belt 24 by 13.3% with respect to its normal length brings the electrode 21 into contact with the dog's axilla with suitable pressure.

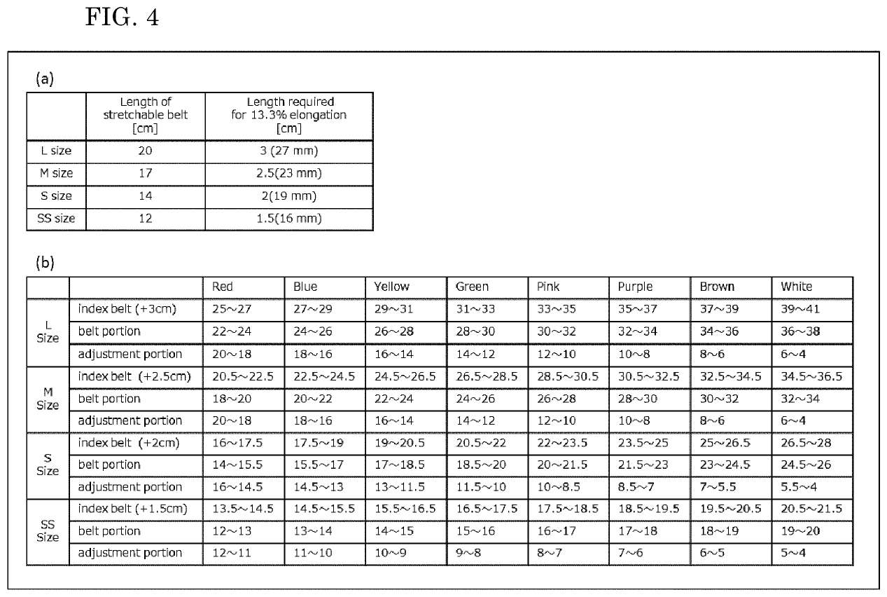

FIG. 4(a) is a table showing the normal length of the stretchable belt 24 for each size of the wearable instrument 1, and also showing, for each size of the wearable instrument 1, length necessary for elongating the stretchable belt 24 by 13.3%. The wearable instrument 1 has four size variations as shown in FIG. 4(b) for instance: size L, size M, size S and size SS. For size L, the length of the stretchable belt 24 is 20 cm; for size M, 17 cm; for size S, 14 cm; and for size SS, 12 cm. In this case, elongating the stretchable belt 24 by 3 cm for size L, by 2.5 cm for size M, by 2 cm for size S, and by 1.5 cm for size SS enables the stretchable belt 24 to be elongated by about 13.3% with respect to its normal length. To make this elongation ratio further close to 13.3%, the stretchable belt 24 needs to be elongated by 27 mm for size L, by 23 mm for size M, by 19 cm for size S, and by 16 mm for size SS.

FIG. 4(b) is a table showing an example of the relationship in length between the small sections 251 to 258 for each size of the wearable instrument 1 and the small sections 701 to 708 of the measuring instrument 2 of corresponding size. The length of the index belt 70 corresponding to each of the small sections 701 to 708 is longer than the length of the belt portion 20 corresponding to each of the small sections 251 to 258, by the length shown in FIG. 4(a).

This enables the ratio for elongating the stretchable belt 24 to be substantially fixed, i.e., 13.3%, in the wearable instrument 1 having any of the sizes. Consequently, the wearable instrument 1 with a size suitably selected in advance enables reduction in excessive tightness and excessive looseness against various body sizes of dogs, and enables the electrodes 21 to come into contact with the dogs with fixed pressure.

It is preferable that the length ratio of each small section to the entire length of the belt portion 20 and the entire length of the index belt 70 be nearly fixed regardless of the size of the wearable instrument 1. Thus, in the example of FIG. 4(b), the lengths of the small sections 251 to 258 and the lengths of the small sections 701 to 708 are different for each size of the wearable instrument 1. To be specific, for size L and size M, the small sections 251 to 258 and the small sections 701 to 708 are each 2 cm long. For size S, the small sections 251 to 258 and the small sections 701 to 708 are each 1.5 cm long. For size SS, the small sections 251 to 258 and the small sections 701 to 708 are each 1 cm long.

### How to Adjust Length of Belt Portion 20

The following describes how to adjust, with the measuring instrument 2, the length of the belt portion 20 of the wearable instrument 1. The first step is attaching the measuring collar 60 to the dog's neck (i.e., attachment step). The next step is passing the index belt 70 through the dog's armpit, followed by placing a second end of the index belt 70 onto the dog's back to check a graduation of the measuring-instrument index 71, the graduation being located in a portion on the index belt 70 intersecting with the outer edge on the side of the collar accessory 80 (i.e., index check step). The following step is adjusting the length of the belt portion 20 on the basis of a graduation of the wearable-instrument index 26 on the belt portion 20, the graduation corresponding to the graduation of the measuring-instrument index 71 checked in the index checking step (i.e., adjustment step).

After adjusting the length of the belt portion 20 through the foregoing procedures, the subsequent step is detaching the measuring instrument 2 from the dog's neck, followed by attaching the collar 10 to the dog's neck, followed by pulling the belt portion 20 up via the dog's right and left armpits to join the joint 22 to the joint 32. This completes the attachment of the wearable instrument 1 to the dog. Attaching the wearable instrument 1 to the dog after adjusting the length of the belt portion 20 in advance, in this way, reduces the possibility of unsuccessful length adjustment, when compared to adjusting the length of the belt portion 20 after attaching the wearable instrument 1 to the dog moving around. It is noted that the user may perform the adjustment step after detaching the measuring instrument 2 from the dog's neck. It is also noted that the user may perform the adjustment step after attaching the collar 10 to the dog's neck or after joining the joint 22 to the joint 32.

FIG. 5(a) and FIG. 5(b) show how the wearable instrument 1 is attached to the dog. FIG. 5(a) is a diagram in the middle of the attachment, and FIG. 5(b) is a diagram after the completion of the attachment. When attaching the wearable instrument 1 to the dog, the user first attaches the collar 10 to the dog's neck without joining the joints 22 to the joints 32. The user then pulls the belt portions 20 up via the dog's armpits and joins the joints 22 to the joints 32. This completes the attachment of the wearable instrument 1 to the dog as shown in FIG. 5(b).

As described above, the wearable instrument 1 includes the collar 10 and the belt portions 20. On each belt portion 20 is indicated an index for length adjustment with the joint 22. Adjusting the position of the joint 22 to a region where a suitable index is displayed achieves fixed elastic force generated by the stretchability of at least part of the belt portion 20. This enables the user to highly reproducibly adjust the pressure exerted on the dog's axilla by the electrode 21 when the wearable instrument 1 is attached to the dog, when compared to, for instance, a wearable instrument for adjusting its belt length with the dog wearing the wearable instrument.

The wearable instrument 1 is configured such that the index indicated on the belt portion 20 includes a plurality of marks displayed in the length direction of the belt portion 20 and having different colors from each other. This configuration enables the user to adjust the length of the belt portion 20 through an intuitive method, color matching.

When the electrode 21 exerts a suitable degree of pressure on the dog's axilla, the ratio of elongation of the stretchable belt 24 varies depending on the material and shape of the stretchable belt 24. The ratio of elongation of the stretchable belt is thus not limited to the aforementioned value, 13.3%. The number and length of small sections on the belt portion 20 and index belt 70, where indexes are shown, are not limited to the foregoing examples.

The size variations of the wearable instrument set are not limited to the foregoing examples. The wearable instrument set may have size variations such as a smaller size than size SS or a larger size than size L. The wearable instrument set may have three kinds or less of size variation, or may have five kinds or more of size variation.

### Second Embodiment

The following describes another embodiment of the present disclosure. The present embodiment describes a measuring instrument 2A, which is another embodiment of the measuring instrument 2. For the sake of convenience in description, components whose functions are the same as those described in the first embodiment are denoted by the same signs and will not be elaborated upon.

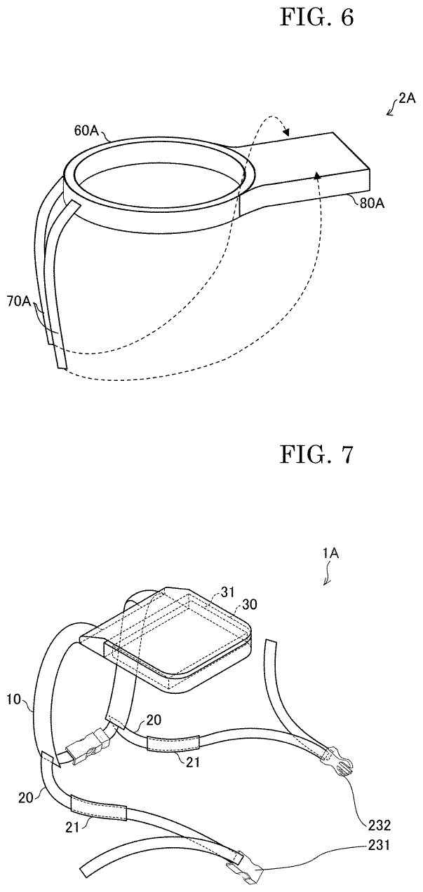

FIG. 6 is a perspective view of the configuration of the measuring instrument 2A according to the present embodiment. As shown in FIG. 6, the measuring instrument 2A includes a measuring collar 60A, index belts 70A, and a collar accessory 80A, instead of the measuring collar 60, index belt 70, and collar accessory 80.

The measuring collar 60A has substantially the same shape as the collar 10 of the wearable instrument 1. The index belts 70A have substantially the same shape as the belt portions 20 of the wearable instrument 1. It is noted that the measuring instrument 2A may include only one index belt 70A, not two index belts 70A. The collar accessory 80A has substantially the same shape as the casing 30 of the wearable instrument 1. That is, the measuring instrument 2A is similar to the wearable instrument 1 in shape when compared to the measuring instrument 2.

For checking a length index using the measuring instrument 2A, such a similar shape is less likely to generate a deviation in an index graduation resulting from the difference in shape between the measuring instrument 2A and the wearable instrument 1. A wearable instrument set according to one aspect of the present disclosure may include the wearable instrument 1 and the measuring instrument 2A.

### Third Embodiment

The following describes another embodiment of the present disclosure. The present embodiment describes a wearable instrument 1A, which is another embodiment of the wearable instrument 1.

FIG. 7 is a perspective view of the configuration of the wearable instrument 1A according to the present embodiment. As shown in FIG. 7, the wearable instrument 1A is different from the wearable instrument 1 in the following regards: the wearable instrument 1A does not include the joints 32; and the wearable instrument 1A includes joints 231 and 232 (i.e., adjustment portions) connectable to each other, instead of the joints 32 connectable to the joints 22.

In the wearable instrument 1A, when the joints 231 and 232 are joined together at the dog's back, the two belt portions 20 form an annular portion between the belt portions 20 and part of the collar 10. The technical scope of the present disclosure encompasses the wearable instrument 1A with such a shape as well. A wearable instrument set according to one aspect of the present disclosure may include the wearable instrument 1A and the measuring instrument 2, or may include the wearable instrument 1A and the measuring instrument 2A.

The present disclosure is not limited to the foregoing individual embodiments. Various modifications can be made within the scope of the claims The technical scope of the present disclosure encompasses an embodiment obtained in combination, as appropriate, with the different technical means disclosed in the different embodiments. Furthermore, combining the technical means disclosed in the individual embodiments can form a new technical feature.

### CROSS-REFERENCE TO RELATED APPLICATION

The present application claims priority to Japanese Patent Application No. 2017-156090, filed Aug. 10, 2017, the contents of which are incorporated herein by reference in its entirety.

### REFERENCE SIGNS LIST

1, 1A wearable instrument

2, 2A measuring instrument

10 collar

20 belt portion

22, 231, 232 joint (adjustment portion)

26 wearable-instrument index (first index)

261 to 268 mark

60, 60A measuring collar

70, 70A index belt

71 measuring-instrument index (second index)

80, 80A collar accessory

1. A wearable instrument for an animal, comprising:
a collar attachable to a neck of the animal; and
a belt portion having one end joined to the collar, the belt portion extending to a back of the animal via an armpit of the animal when the wearable instrument is attached to the animal,
wherein the belt portion includes an adjustment portion where a length of the belt is adjusted,
the belt portion is at least partly stretchable,
the belt portion includes a first index indicated for the length adjustment with the adjustment portion, and
the first index corresponds to a second index indicated on an index belt included in a measuring instrument corresponding to the wearable instrument.

2. The wearable instrument according to claim 1, wherein the first index includes a plurality of marks displayed in a length direction of the belt portion.

3. The wearable instrument according to claim 2, wherein the plurality of marks have colors different from each other.

4. A measuring instrument comprising:
a measuring collar attachable to a neck of an animal;
an index belt having a first end joined to the measuring collar, the index belt including a second index indicated in accordance with a distance from a joint between the measuring collar and the index belt; and
a collar accessory joined to the measuring collar, the collar accessory being disposed at a back of the animal when the measuring instrument is attached to the animal.

5. A wearable instrument set comprising:
the wearable instrument according to claim 1; and
wherein the first index indicated on the belt portion corresponds to the second index indicated on the index belt.

6. A method for adjusting the length of the belt portion of the wearable instrument according to claim 1, the method comprising:
an attachment step of attaching the collar to the neck of the animal;
an index check step of passing the index belt through an armpit of the animal, followed by placing a second end of the index belt onto a back of the animal to check a graduation of the second index located on a portion of the index belt intersecting with a collar accessory; and
an adjustment step of adjusting the length of the belt portion, based on a graduation of the first index on the belt portion, the graduation of the first index corresponding to the graduation of the second index checked in the index check step.
